(19) United States
(12) Patent Application Publication
(10) Pub. No.: US 2021/0302432 A1
(43) Pub. Date: Sep. 30, 2021

(54) MONITORING MYCOTOXINS IN THE BLOOD OF PIGS OR BROILER CHICKENS

(71) Applicants: Innov Ad N.V., Essen (BE); Universiteit Gent, Gent (BE)

(72) Inventors: Milena Sevastiyanova, Essen (BE); Christos Gougoulias, Essen (BE); Ben Letor, Essen (BE); Marianne Lauwers, Merelbeke (BE); Siska Croubels, Merelbeke (BE); Mathias Devreese, Merelbeke (BE); Siegrid De Baere, Merelbeke (BE)

(21) Appl. No.: 16/821,704

(22) Filed: Mar. 17, 2020

(51) Int. Cl. G01N 33/68; G01N 30/06; G01N 30/88

(52) U.S. Cl. CPC G01N 33/6848; G01N 2030/8822; G01N 30/88; G01N 30/06

(57) ABSTRACT

The present invention relates to a method for the detection of mycotoxins and their phase I and phase II metabolites in broiler chickens and pigs, the method comprising:
collecting the blood of broiler chickens and pigs as a dried blood sample;
preparing the dried blood sample for analysis;
analyzing the prepared dried blood sample by liquid chromatography-tandem mass spectrometry and high-resolution mass spectrometry.
   Such method can advantageously be used for the assessment of the exposure of pigs or broiler chickens to feed contaminated with mycotoxins. Also, such method can be used for assessing the impact of the addition of mycotoxin detoxifying agents to animal feed.

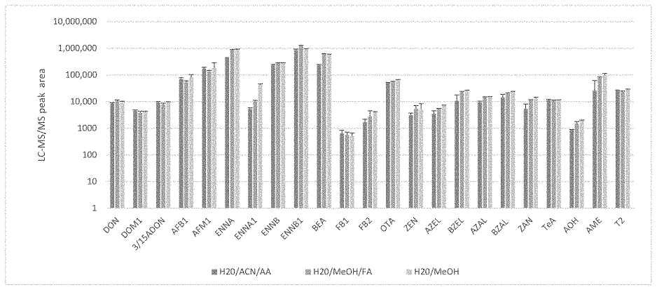

### FIELD OF THE INVENTION

The present invention relates to a novel and inventive method for enhancing productivity in the agricultural business. More in particular, the present invention relates to a novel and inventive method for biomonitoring mycotoxins and their phase I and phase II metabolites in an easy and user-friendly manner via accessible animal matrices, and more specifically in the blood of broiler chickens or pigs.

### BACKGROUND PRIOR ART OF THE INVENTION

The need for increased protein consumption via meat production in a sustainable manner globally, is directly linked with the need for reduction of the overall production cost. This certainly remains the primary objective within the pig and broiler chicken industry with the feed cost having the biggest impact. The high and unpredictable variability of raw material costs (cereals, proteins and fats) and the ever growing and complex load of toxins contamination continue to negatively impact productivity.

Several stress factors such as handling, sudden environmental changes, heat, feed programs & diet composition changes, vaccination, disease challenges, infections and impaired immune response are affecting animal health and productivity. Modern farming animals possess a limited natural resistance and immunity against such stresses leading often to oxidative stress, when the animal is no longer capable of detoxifying timely the reactive oxygen species at cell level. The impact of such stress factors is worsened and amplified in the presence of mycotoxins.
Mycotoxins decrease the function of key metabolic organs such as the liver and kidneys.
Mycotoxins have a significant negative impact on the poultry and swine defense mechanisms and immune system.
For example, fumonisins & deoxynivalenol (DON) predispose to the development of necrotic enteritis in broilers.

In today's environment, the presence of mycotoxins is an inherent risk. Chances are high that multiple mycotoxigenic molds such as Aspergillus, Fusarium and Penicillium and the related toxins creep into silos and corn silage at harvest and after ensiling. A recent survey highlighted that 99% of grain samples collected contained mycotoxins and over 85% contained multiple mycotoxins. More than 400 types of mycotoxins have been identified so far, also diverse in their chemistry and effect on animals. Feed contaminated with mycotoxins causes a broad spectrum of problems ranging from reduction in feed intake and growth performance to compromised reproduction, health and immunity. Symptoms are often non-specific and cost the agricultural sector billions of dollars per annum.

Feed dietary mycotoxins can also end up in animal products destined for human consumption such as milk, eggs and meat where they remain as stable and inert toxic molecules but their fate is unknown once within the human body.

Under normal conditions multiple-mycotoxin contamination is likely. Multiple mycotoxins can have a synergistic or additive negative effect, increasing the overall negative impact on animal's performance and health. Mycotoxins from fungi in feed combined with bacterial toxins further increase the negative health issues.

Mycotoxins may also occur in a conjugated form either soluble (masked or modified mycotoxins produced by fungi and/or plants) or incorporated onto/associated with/attached to macromolecules (bound mycotoxins). That effectively means that not only ‘parent’ mycotoxins but also their fungal and plant metabolites need to be quantified precisely for an appropriate feed risk assessment. However, individual raw material (grains/seeds) and/or feed sampling for mycotoxin analysis, although a critical factor, is often overlooked due to the high cost and the relative long (analysis) time involved. Moreover, the sampling itself of raw materials constitutes a major limitation due to the existence of what is known as ‘hot’ spots and the estimated relevant error occurrence during the sampling procedure is ˜88%. Additionally, mycotoxin analysis of the raw materials does not necessarily warrant a true representation of the final feed that the animal consumes, as more mycotoxin transformations may take place during storage. Also, producers often rely on quick and often cheap(er) analysis tools for mycotoxin detection such as Lateral Flow Devices (LFDs) and ELISAs. While most of the LFDs provide qualitative results, some more recent versions of LFDs and all ELISAs provide quantitative results. However, both methods are limited in the fact that a) cross-reactivity may occur which in turn can invalidate the results and thereby, impact scientific reproducibility and that b) only one mycotoxin can be detected at a time and that not all tests are fit for different types of feed. Thus, this approach proves either insufficient or uneconomical for a full feed risk assessment.

More importantly, inherently, the feed risk assessment approach per se does not provide a true representation of the true exposure of animals to mycotoxins. Only methodological approaches that aim/allow a) the detection of several mycotoxins and their Phase I and Phase II metabolites b) with high analytical precision, c) in biological fluids in animals and d) with a practical, user-friendly as well as easy to sample and transport method can provide such meaningful information. It is only then when someone can get a true representation of the mycotoxins and their metabolites that systemically enter and expose the animal.

Reference is hereby made to a Research Paper entitled “Multi-mycotoxin analysis using dried blood spots and dried serum spots” by B. Osteresch, S. Viegas, B. Cramer and H-U Humpf in Anal. Bioanal. Chem (2017) 409:3369-3382; (Osteresch et al. 2017)

This study discloses a multi-mycotoxin approach for biomonitoring and quantification of 27 important mycotoxins and mycotoxin metabolites in human blood samples.
For a.o. Dried Blood Spots (DBS), HPLC-MS/MS detection method is used.
The study establishes a validated multi-mycotoxin approach for the detection of 27 mycotoxins and metabolites in dried blood/serum spots based on a fast sample preparation followed by sensitive HPLC-MS/MS analysis.

However, this study deals exclusively with the application of the methodology to human applications and transfer of the proposed methodology to animals cannot be taken for granted.

In a first paragraph, in the introduction pg. 3370, it is stated that Dried Blood Spots (DBS) are suitable for extensive biomonitoring studies of environmental contaminants in humans or animals. More specifically, reference is made to study nr. 14 by Batterman S, Chernyak S, entitled “Performance and storage integrity of dried blood spots for PCB, BFR and pesticide measurements”, Sci Total Environ. 2014; 494-495:252-60. This study investigates the suitability of DBS analyses for population studies of exposure to three chemical groups: polychlorinated biphenyls (PCBs), brominated flame retardants (BFRs), and chlorinated pesticides.

The collection of whole blood samples on paper, known as dried blood spot (DBS), dates back to the early 1960s in human newborn screening for inherited metabolic disorders. The last five years, DBS-LC-MS/MS has emerged as an important method for quantitative analysis of several molecules/analytes. The DBS-LC-MS/MS approach has been expanded for different types of analytes in the human sector such as polychlorinated biphenyls (PCBs), Brominated flame retardants (BFRs), pesticides (Batterman S and Chernyak S, 2014), hormones (Thomas and Thevis, 2018) and drugs (Wickremsinhe et al. 2018). However, the DBS-LC-MS/MS approach has not, yet, been adopted within the veterinary sector and no prior application for the detection of mycotoxins in farming animals is known to date. Several parameters need to be checked and validated for appropriate adoption on a particular animal species such as analytical sensitivity due to the small sample size, knowledge of the animal species-specific in vivo metabolism of mycotoxins to determine the most appropriate biomarker, potential impact of various blood sample properties on accurate quantification, sample quality, the impact of blood hematocrit, analyte stability, within day analytical variation, between days analytical variation etc.

In that context, it should be noted that this study by Batterman S, Chernyak S (2014) referenced in the introduction of this Research Paper refers to the detection of pesticides and other environmental toxic contaminants via DBS in animals, but not per se to mycotoxins. The transfer of the DBS concept for the detection of the environmental contaminants such as pesticides and heavy metals to mycotoxins, is neither simple nor evident or obvious.

In support hereof, it should be noted that PCBs, BFRs, and several classes of manmade chemicals such as pesticides are typically ‘organo-synthetic’ structures. Namely not naturally occurring structures as they carry one or more chlorine, bromine, fluorine (or other) atoms of non-naturally occurring elements within organic based structures.

As a result, not only their corresponding physicochemical properties but also their stability in DBS by application in paper based Find The Agent (FTA) cards may be totally different to that of mycotoxins, the latter being solely naturally occurring (organic) molecules. In fact, the authors Batterman & Chernyak, report that the DBS method proved stable at room temperature for pesticides and PCBs but not for Polybrominated Diphenyl ethers (PBDEs).

Differently phrased, one needs to perform substantive research efforts in order to determine:
which mycotoxins and their specific (Phase I & Phase II) metabolites should be targeted and bio-monitored;
for a specific animal species and;
for a specific biological sample (urine, blood, feces, excreta etc.);
   as this relates directly to their unique metabolic pathway for a specific animal species.

Further, even if satisfactorily responses to the above questions are at hand, there still remains the need for a reliable, cost-effective means and method so as to detect in a qualitative and quantitative manner the presence of such mycotoxins and its metabolites in a sample of the selected animal to be bio-monitored.

Until today, mycotoxins are determined in the animal production farming field only in feed. Feed analysis provides only a rough estimate of the risk involved in relation to the amount of mycotoxins that animals could be exposed to. Moreover, feed analysis is prone to significant methodological errors due to the presence of hotspots and the difficulty of determining masked mycotoxins, both leading to underestimation of the risk. Therefore, although the feed risk assessment still remains a tool, its real usefulness, when applied in isolation, remains highly questionable as it lacks vital information with regards to the true exposure of animals to mycotoxins.

Additionally, routine mycotoxin biomonitoring methods do not include mycotoxin phase I and phase II metabolites. This may significantly underestimate mycotoxin exposure especially for heavily metabolized mycotoxins. Additional research efforts are also needed to measure metabolites in vivo after exposure and to establish which mycotoxin metabolites should be prioritized for the inclusion during biomonitoring efforts.

As a result, there remains a clear need for a cost effective and reliable method for determining the true exposure of mycotoxins to animals, broiler chicken and pigs in particular.

### REFERENCES

Batterman S, Chernyak S Performance and storage integrity of dried blood spots for PCB, BFR and pesticide measurements, Sci Total Environ. 2014; 494-495:252-60
Thomas A, Thevis M, Analysis of insulin and insulin analogs from dried blood spots by means of liquid chromatography-high resolution mass spectrometry. Drug Testing and Analysis. 2018; doi.org/10.1002/dta.2518
Lauwers, Marianne et al. 2019. “Multi LC-MS/MS and LC-HRMS Methods for Determination of 24 Mycotoxins Including Major Phase I and II Biomarker Metabolites in Biological Matrices from Pigs and Broiler Chickens.” Toxins 11(3): 171.
Osteresch, Bernd, Susana Viegas, Benedikt Cramer, and Hans Ulrich Humpf. 2017. “Multi-Mycotoxin Analysis Using Dried Blood Spots and Dried Serum Spots.” Analytical and Bioanalytical Chemistry 409(13): 3369-82. http://www.ncbi.nlm.nih.gov/pubmed/28299415 (Feb. 13, 2020).
Wickremsinhe, Enaksha R. et al. 2018. “Incorporating Dried Blood Spot LC-MS/MS Analysis for Clinical Development of a Novel Oncolytic Agent.” Bioanalysis 10(5): 341-56.

### OBJECTIVES OF THE INVENTION

The aim of the inventors is to find novel ways to evaluate the systemic exposure of feed mycotoxins and their intermediates on animals.

Routine mycotoxin biomonitoring methods do not include mycotoxin phase I and phase II metabolites and this may significantly underestimate mycotoxin exposure especially for heavily metabolized mycotoxins.

The primary aim and objective of the invention is to provide a) adequate methods and b) cheap, easy and user-friendly means that focus on animal mycotoxin exposure assessment.

In that context, the main objectives of the invention are to identify novel biomarkers of exposure and to provide appropriate analytical tools for the precise determination and quantification not only of mycotoxins but also of their relevant metabolites in vivo after exposure and to establish which mycotoxin metabolites should be prioritized for the inclusion during biomonitoring efforts for chickens or pigs.

A further objective of the invention is that such biomonitoring method and means may provide more reliable data related to the animal's true impact via contaminated feed and in combination with a number of biotic and abiotic stress factors (described previously) in situ i.e. under real farming conditions.

It is thus, believed that the invention has a great potential for use as a diagnostic tool with significant economic impact in industrial animal farming.

These objectives and advantages are given only by way of illustrative example, and such objectives may be exemplary of one or more embodiments of the invention. Other desirable objectives and advantages inherently achieved by the disclosed invention may occur or become apparent to those skilled in the art.

In view of the above, there remains a need for a reliable and cost-efficient biomonitoring method for determining the exposure of animals to multi-mycotoxins, in particular broiler chicken or pigs.

### STATEMENT OF INVENTION

The present inventors have conducted extensive studies in order to solve the above-mentioned problems.

These studies have resulted in the invention as described hereinafter.

The inventors have successfully found a method for biomonitoring mycotoxins and their relevant phase I and phase II metabolites in the blood of broiler chickens or pigs as described hereinafter.

The invention is set forth and characterized in the main claim, while the dependent claims describe other characteristics and specific features for preferred embodiments of the invention.

According to one aspect of the invention, there is provided a method for the detection of one or more mycotoxins and one or more of their phase I and/or one or more of their phase II metabolites in broiler chickens or pigs, the method comprising:
collecting the blood of broiler chickens or pigs as a dried blood sample;
preparing the dried blood sample for analysis;
analyzing the prepared dried blood sample by liquid chromatography-tandem mass spectrometry (LC-MS/MS) and high resolution mass spectrometry (HRMS).

The term ‘detection’ in the context of the present invention should be understood as on the one hand the (qualitative) determination of the presence of certain compounds and—at least as the mycotoxins and some of their phase I metabolites are concerned—on the other hand the (quantitative) calculation of the concentration of these compounds. According to a preferred embodiment of the invention, the compounds subject of the detection according to the method of the present invention are some specified mycotoxins and some of their phase I and phase II metabolites, as defined hereinafter.

According to a preferred embodiment of the invention, such method is directed to the detection of one or more mycotoxins selected from the following list of 29 mycotoxins and their phase I and phase II metabolites:

Deoxynivalenol, De-epoxy-deoxynivalenol, 3/15-acetyldeoxynivalenol, T2-toxin, HT-2 toxin, Aflatoxin B1, Aflatoxin M1, Ochratoxin A, Enniatin A1, Enniatin A, Enniatin B, Enniatin B1, Beauvericin, Fumonisin B1, Fumonisin B2, Tenuazonic acid, Alternariol, Alternariol methyl ether, Zearalenone, Zearalanone, α-Zearalenol, α-Zearalanol, β-Zearalanol, β-Zearalenol, DON-glucuronide, DON-sulphate, ZEN-glucuronide, α-ZEL-glucuronide and β-ZEL-glucuronide. (note: the 3/15-acetyldeoxynivalenol are in fact two different compounds, namely 3-acetyldeoxynivalenol and 15-acetyldeoxynivalenol; in the method of the present invention, both these compounds give rise to one and the same retention time and the same molecular mass; therefore, in the context of detection by the method of the present invention, they are considered as one and the same compound.)

According to a further preferred embodiment of the invention, the method comprises

the detection of:
one or more mycotoxins selected from the following list of 17 parent mycotoxins:
   Deoxynivalenol,
   3/15-acetyldeoxynivalenol,
   T2-toxin,
   HT-2 toxin,
   Aflatoxin B1,
   Ochratoxin A,
   Enniatin A1,
   Enniatin A,
   Enniatin B,
   Enniatin B1,
   Beauvericin,
   Fumonisin B1,
   Fumonisin B2,
   Tenuazonic acid,
   Alternariol,
   Alternariol methyl ether,
   Zearalenone,
one or more phase I metabolites selected from the following list of 7 phase I metabolites:
   De-epoxy-deoxynivalenol,
   Aflatoxin M1,
   α-Zearalenol,
   α-Zearalanol,
   β-Zearalanol,
   β-Zearalenol,
   Zearalanone,
one or more phase II metabolites selected from the following list of 5 phase II metabolites:
   Deoxynivalenol-glucuronide,
   Deoxynivalenol-sulphate,
   Zearalenone-glucuronide,
   α-zearalenol-glucuronide,
   β-zearalenol-glucuronide.

With ‘parent’ mycotoxin is meant the mycotoxin that can be found in the feed, this to make the difference with a metabolite of such mycotoxin that can be formed in the animal (for example in the liver).

According to a further preferred embodiment of the invention, collecting the dried blood sample comprises collecting a drop of blood on a filter paper, followed by drying at room temperature.

According to a further preferred embodiment of the invention, collecting the dried blood sample comprises isolating the dried blood sample from the filter paper by punching out a paper disk out of the filter paper, preferably round and about 8 mm in diameter, using a biopsy punch.

According to a further preferred embodiment of the invention, prior to analyzing, the blood from the dried blood sample is prepared for analysis, such preparation including extracting the dried blood sample in an extraction solvent.

According to a further preferred embodiment of the invention, the extraction solvent comprises a water/acetonitrile/acetone mixture.

According to a further preferred embodiment of the invention, the extraction solvent is dried and reconstituted in a reconstitution solvent.

According to a further preferred embodiment of the invention, the reconstituting solvent comprises a water/methanol/formic acid mixture.

According to a further preferred embodiment of the invention, the collected dried blood sample is extracted in an extraction solvent, dried, reconstituted in a reconstitution solvent, whereupon the reconstituted dried blood sample is analyzed by liquid chromatography tandem mass spectrometry.

The term Mass Spectrometry is hereinafter referred to as MS.

The term tandem Mass Spectrometry is hereinafter referred to as MS/MS.
The term High Resolution Mass Spectrometry is hereinafter referred to as HR-MS.
The term Liquid Chromatography is hereinafter referred to as LC.

According to a further preferred embodiment of the invention, the method comprises analyzing the reconstituted dried blood sample in a two subsequent steps:
on the one hand, analyzing by LC-MS/MS, the mass spectrometer apparatus operating in negative electro-spray ionization mode, and using as mobile phase, a mixture of acetic acid, water and acetonitrile, the proportion of acetonitrile gradually increasing during the liquid chromatography process, one or more of the following mycotoxins and phase I metabolites: zearalenone (ZEN), zearalanone (ZAN), α-zearalenol (AZEL), α-zearalanol (AZAL), β-zearalanol (BZAL), β-zearalenol (BZEL), tenuazonic acid (TEA), alternariol (AOH), alternariol methyl ether (AME);
on the other hand, analyzing by LC-MS/MS, the mass spectrometer apparatus operating in positive electro-spray ionization mode, and using as mobile phase, a mixture of ammonium formate, formic acid, water and methanol, the proportion of methanol gradually increasing during the liquid chromatography process, one or more of the following mycotoxins and phase I metabolites:
deoxynivalenol (DON), de-epoxy-deoxynivalenol (DOM1), 3/15-acetyldeoxynivalenol (3/15ADON), T2-toxin (T2), HT-2 toxin (HT2), aflatoxin B1 (AFB1), aflatoxin M1 (AFM1), ochratoxin A (OTA), enniatin A1 (ENNA1), enniatin A (ENNA), enniatin B (ENNB), enniatin B1 (ENNB1), beauvericin (BEA), fumonisin B1(FB1) and fumonisin B2 (FB2).

The method of the invention further comprises detection of some phase II metabolites of such mycotoxins either by LC-MS/MS (if analytical standards are commercially available, e.g. DON-glucuronide, ZEN-glucuronide) or by LC-HRMS.

According to a further preferred embodiment of the present invention, the method comprises combining any of the abovementioned preferred embodiments of the method of the invention.

According to a further preferred embodiment of the present invention, the method comprises spiking the collected dried blood sample with one or more internal standards.

According to further preferred embodiment of the present invention, the internal standard(s) is, resp. are selected from the following list of compounds:

¹³C₁₅-deoxynivalenol, ¹³C₁₇-aflatoxin B1, ¹³C₂₀-ochratoxin A, ¹³C₂₄-T2-toxin, ¹³C₃₄-fumonisin B1, ¹⁵N₃-enniatin B, ¹³C₆¹⁵N-tenuazonic acid, ¹³C₁₈-zearalenone.

The method as set forth above, as well as any of the preferred embodiments of such method, can be used for the assessment of the exposure of pigs and broiler chickens to feed contaminated with mycotoxins.

Further such method is also suitable for assessing the addition of mycotoxin detoxifying agents to animal feed.

Further aspects and advantages of the invention as well as of its preferred embodiments will appear from the following detailed description.

### BRIEF DESCRIPTION OF THE DRAWING

The FIGURE shows the evaluation of the analyte peak areas after LC-MS/MS of DBS (60 μl).

### DETAILED DESCRIPTION OF THE INVENTION

In order for the method of the present invention to be suitable for practical use, the various steps comprised in such method need to be validated. To that end, various steps comprised within such method have been tested by the present inventors and its results have been published in the following two articles. As such these articles describe various aspects of the invention, subject of the present application:
1) the Article entitled “Multi LC-MS/MS and LC-HRMS Methods for Determination of 24 Mycotoxins including Major Phase I and II Biomarker Metabolites in Biological Matrices from Pigs and Broiler Chickens”, by Marianne Lauwers, Siegrid De Baere, Ben Letor, Michael Rychlik, Siska Croubels and Mathias Devreese, published on Mar. 19, 2019, by Toxins 2019, 11, 171; doi: 10.3390/toxins 11030171, www.mdpi.com/journal/toxins, hereinafter referred to as Article (1);
2) The Article entitled “Assessment of dried blood spots for multi-mycotoxin biomarker analysis in pigs and broiler chickens” by Marianne Lauwers, Siska Croubels, Siegrid De Baere, Milena Sevastiyanova, Eva Maria Romero Sierra, Ben Letor, Christos Gougoulias and Mathias Devreese, published on Sep. 18, 2019 by Toxins 2019, 11, 541; doi:10.3390/toxins 11090541, www.mdpi.com/journal/toxins, hereinafter referred to as Article (3).

In the detailed description that follows, for some aspects of the invention, we will refer to the contents of these articles. To the extent the contents of these articles have not been explicitly reproduced in the present specification, they are incorporated hereinto by reference.

In the detailed description that follows hereinafter, reference is made to the above articles as Article (1) and Article (2) respectively.

In document (1) the necessary Method Development and the validation of the appropriate ‘Mycotoxin biomarkers for exposure’ in several biological fluids (blood plasma, urine, feces and excreta) in pigs and chicken was developed.

In document (2) the transfer of the Analytical (detection) methodology from (liquid) blood plasma to dried blood spots (hereinafter referred to as DBS) both for mycotoxins and phase I and phase II metabolites, was statistically validated.

As said supra, the present invention relates to a method for monitoring or assessing mycotoxins and their phase I and phase II metabolites in the blood of broiler chickens and pigs. Such method comprises the following two steps:
sampling the blood by collecting dried blood samples;
preparing the dried blood sample for analysis;
analyzing the dried blood samples.

The term “monitoring” as used in the present specification is understood to mean determining the exposure of animals to mycotoxins with the use of so-called biomarkers of exposure.

The term “biomarkers” as used in the present specification is understood to main molecules that are a measure for the exposure by the animal to the mycotoxins and their phase I and phase II metabolites, and that can be found in the biological matrices of the animals.

Two types of biomarkers should be distinguished:
direct (exposure) and
indirect (mechanism/effect) biomarkers.

The latter are mainly non-specific and associated with either the effect or the mechanism of mycotoxins. The effect-based biomarkers are even less specific than the mechanism-based. A typical example is the alteration in feed intake after administration of deoxynivalenol. The direct biomarkers are specific and directly linked to the exposure. This type of biomarker is often the mycotoxin itself or their phase I and II metabolites. In biomonitoring, the direct biomarkers are the preferred biomarkers.

The detection of biomarkers can occur in easily accessible matrices of the animal to be bio-monitored, such as blood, urine or feces.

In the context of the present invention, detection of the direct biomarkers or biomarkers for exposure, for example for rapidly absorbed mycotoxins, are to be detected in the blood.

Peak concentrations can be detected in the blood and the concentration in blood is directly related with the exposure. Plasma is thus a good matrix to measure mycotoxins a few hours after exposure.

The time at which mycotoxins are absorbed by the animal varies substantially between the various mycotoxins and between animal species. The inventors have determined after experiments that the optimal time slot for the detection of such mycotoxins, is to collect the dried blood sample after 30 minutes up to two hours after feeding, preferably approximately 1 hour after feeding the animal.

The experimental data for this preferential time frame are described in the following article by the inventors:

The Article entitled “Biomarkers for Exposure as a tool for efficacy testing of a mycotoxin detoxifier in broiler chickens and pigs”, by Marianne Lauwers, Siska Croubels, Ben Letor, Christos Gougoulias and Mathias Devreese, published on Mar. 28, 2019 by Toxins 2019, 11, 187; doi:10.3390/toxins 11040187, www.mdpi.com/journal/toxins.

The term “Liquid Chromatography (LC)” as used in the context of the present invention should be understood as a laboratory technique for the separation of a mixture of compounds. The mixture is dissolved in a fluid called the mobile phase, which carries it through a structure holding another material called the stationary phase. The various constituents of the mixture travel at different speeds, causing them to separate. The separation is based on differential partitioning between the mobile and stationary phases. Subtle differences in a compound's partition coefficient result in differential retention on the stationary phase and thus affect the separation.

Chromatography may be preparative or analytical. The purpose of preparative chromatography is to separate the components of a mixture for later use, and is thus a form of purification. Analytical chromatography is done with smaller amounts of material and is for establishing the presence or measuring the relative proportions of analytes in a mixture. For the purpose of the present invention, analytical chromatography is applied.

The term “Mass Spectrometry (MS)” as used in the context of the present invention should be understood as an analytical technique that measures the mass-to-charge ratio of ions. The results are typically presented as a mass spectrum, a plot of intensity as a function of the mass-to-charge ratio. In the present case, mass spectrometry is applied to a quite complex organic mixture.

A mass spectrum is a plot of the ion signal as a function of the mass-to-charge ratio. The spectra are used to determine the elemental or isotopic signature of a sample, the masses of particles and of molecules, and to elucidate the chemical identity or structure of molecules and other chemical compounds.

In the MS procedure as applied in the present invention, a sample, being the collected dried blood sample, extracted, dried and reconstituted as described below, is ionized. This will cause some of the sample's molecules to break into charged fragments or simply become charged without fragmenting. These ions are then separated according to their mass-to-charge ratio, for example by accelerating them and subjecting them to an electric or magnetic field: ions of the same mass-to-charge ratio will undergo the same amount of deflection. The ions are detected by a mechanism capable of detecting charged particles, such as an electron multiplier. Results are displayed as spectra of the signal intensity of detected ions as a function of the mass-to-charge ratio. The molecules in the dried blood sample are then identified either through a characteristic pattern for the whole of the mycotoxin or phase I metabolite molecules or through a characteristic pattern for the fragments of the mycotoxin or metabolite molecules.

The term “Liquid Chromatography tandem Mass Spectrometry (LC MS/MS)” as used in the context of the present invention should be understood as Liquid Chromatography, followed by Mass Spectrometry. In such a case the Dried Blood Sample, after being extracted in the extraction solvent, being dried and reconstituted in the reconstituting solvent, is injected in the column of the Liquid Chromatography apparatus; the various mycotoxins and phase I metabolites, exiting the Liquid Chromatography column, are then subjected to an identification and quantification analysis by a Mass Spectrometry apparatus, based on a targeted approach.

The term “High Resolution Mass Spectrometry (HRMS)” as used in the context of the present invention should be understood as similar to the above-mentioned Mass Spectrometry, except that the molecules are identified by correlating known exact masses to the identified accurate masses of the fragments of the mycotoxin molecules. This technique uses a non-targeted approach and applies in particular to the phase II metabolites of parent mycotoxins for which no analytical standards are currently available.

### Mycotoxins and Phase I & II Metabolites:

It is known to the person skilled in the art that animal feed may comprise a quite high number of different kinds of mycotoxins and the related metabolites.

As a first step, comprised in the method of the present invention, a selection should be made as to which mycotoxins and related metabolites should be the subject of the monitoring or assessment operation.

The mycotoxins targeted in the multi-method of the present invention comprise the regulated groups, i.e. aflatoxins, ochratoxin A and several Fusarium mycotoxins and two groups of unregulated or emerging mycotoxins, i.e. Alternaria mycotoxins and selected Fusarium mycotoxins (enniatins and beauvericin).

The multi-method according to the present invention targets the following 24 mycotoxins and the following 5 phase II metabolites:

### 24 Mycotoxins and Phase I Metabolites (and Corresponding Accurate Mass):

| | Theoretical accurate |
|---|---|
| Name | mass (g/mol) |
| Deoxynivalenol (DON) | 296.1260 |
| De-epoxy-deoxynivalenol (DOM1) | 280.1311 |
| 3/15-acetyldeoxynivalenol ( 3/15ADON) | 338.1366 |
| T2-toxin (T2) | 466.2203 |
| HT-2-toxin (T2) | 424.2097 |
| Aflatoxin B1 (AFB1) | 312.0634 |
| Aflatoxin M1 (AFM1) | 328.0538 |
| Ochratoxin A (OTA) | 403.0823 |
| Enniatin A1 (ENNA1) | 667.4408 |
| Enniatin A (ENNA) | 681.4564 |
| Enniatin B (ENNB) | 639.4095 |
| Enniatin B1 (ENNB1) | 653.4251 |
| Beauvericin (BEA) | 783.4095 |
| Fumonisin B1 (FB1) | 721.3885 |
| Fumonisin B2 (FB2) | 705.3936 |
| Tenuazonic acid (TEA) | 197.1052 |
| Alternariol (AOH) | 258.0528 |
| Alternariol methyl ether (AME) | 272.0685 |
| Zearalenone (ZEN) | 318.1467 |
| Zearalanone (ZAN) | 320.1624 |
| α-Zearalenol (AZEL) | 320.1624 |
| α-Zearalanol (AZAL) | 322.1780 |
| β-Zearalanol (BZAL) | 322.1780 |
| β-Zearalenol (BZEL) | 320.1624 |

Near to the name of each mycotoxin, the above table comprises also the name in abbreviated form (such as e.g. DON for Deoxynivalenol). In the description that follows, reference to the above mycotoxins will be made by referring to such abbreviated name.

### 5 Phase II Metabolites

| Name | Theoretical accurate mass (g/mol) |
|---|---|
| Deoxynivalenol-glucuronide | 472.1581 |
| Deoxynivalenol-sulphate | 376.0828 |
| Zearalenone-glucuronide | 494.1788 |
| α/β-zearalenol-glucuronide | 496.1945 |

The selection of the abovementioned mycotoxins and their phase I & II metabolites has been based on a careful study of the regulations as presently in force in the European Union, and the occurrence data in animal feed.

EFSA (European Food Safety Agency)-regulated mycotoxins in feed are AFB1, OTA, FB1+FB2, T2+HT2, ZEN and DON.

Non-regulated mycotoxins are e.g. ENNs, BEA, AOH, AME and TEA.

The mycotoxins and some of the phase I metabolites have been quantified in blood samples of pigs and broiler chickens by LC-MS/MS. Some Phase I and the phase II metabolites were determined by LC-HRMS.

The above tables also show the molecular weight of the substances to be determined in the method of the present invention; these data are used in the HRMS step comprised in the method of the present invention.

We will now describe the various steps of the method according to the invention:

### Production of the Dried Blood Sample.

The present invention is based on the use of Dried Blood Samples, given its convenience in production and ease of conservation and transport.

The Dried Blood Sample is produced by taking a drop of blood sample, transferring same to a saver card (paper card), preferably followed by punching out the central 8 mm disk of the card.

The drop of blood can be taken from any part of the pig or broiler chicken, but preferably the blood is taken from the ear of the pig or from the leg of the broiler chicken.

As a next step, the drop of blood is transferred to a saver card.

As saver card, use can be made e.g. of a protein saver card marketed under the brand name “Whatman 903®”, available from Sigma-Aldrich.

A sample collection area of the 903 Protein Saver Card contains five half-inch circles. Each circle holds 50 to 80 μl of sample.

The optimal volume to cover a complete circle on the saver card was determined as 60 μl. On the saver card, the drop of blood spreads out over the card and fills a circle of at least 8 mm. As a next step, the 8 mm central disk on the saver card is punched out with a biopsy punch.

The so punched out saver paper comprising (part of) the drop of blood, constitutes the dried blood sample hereinafter referred to as the DBS.

Based on the following experiments, it appears that irrespective of the specific amount of the drop of blood taken yields the same results in terms of qualification and quantification of the mycotoxins are obtained.

### Insertion of Internal Standards (IS) in the DBS

At this stage in the method according to the present invention, substances suitable to act as internal standard products are spiked into the DBS.

More in particular, according to a preferred mode of operation of the present invention, a combination of the following substances can be used as such internal standards and to that end are spiked into the DBS:

| Name | Theoretical accurate mass (g/mol) |
|---|---|
| ¹³C₁₅-Deoxynivalenol | 311.1763 |
| ¹³C₁₇-Aflatoxin B1 | 329.1204 |
| ¹³C₂₀-Ochratoxin A | 423.1494 |
| ¹³C₂₄-T2-toxin | 490.3008 |
| ¹³C₃₄-Fumonisin B1 | 755.5025 |
| ¹⁵N₃-Enniatin B | 642.4006 |
| ¹³C₆¹⁵N-Tenuazonic acid | 207.1387 |
| ¹³C₁₈-Zearalenone | 336.2071 |

The abovementioned substances are suited to act as internal standards in the method of the present invention as they comprise either carbon atoms comprising 13 neutrons or nitrogen atoms comprising 15 neutrons and are as such easily detectable by the mass spectrometer and have very similar characteristics as the ¹²C and ¹⁴N components. These substances are used as reference standards in the LC-MS/MS apparatus and in the HRMS apparatus used in the present invention.

Since an isotopically labeled Internal Standard (hereinafter referred to as IS) for each single mycotoxin is too expensive and not commercially available, an IS labeled with [¹³C] or [¹⁵N] was used for each group of mycotoxins, as follows:
[¹³C₁₅]-deoxynivalenol was used as IS for DON, DOM1 and 3/15ADON;
[¹³C₂₄]-T2-toxin for T2 and HT2;
[¹³C₁₇]-Aflatoxin B1 for AFB1 and AFM1;
[¹³C₂₀]-Ochratoxin A for OTA;
[¹³C₃₄]-Fumonisin B1 for FB1 and FB2;
[¹³C₆¹⁵N]-Tenuazonic acid for TEA, AME and AOH;
[¹³C₁₈]-Zearalenone for ZEN, AZAL, BZAL, AZEL, BZEL and ZAN; and
[¹⁵N₃]-Enniatin B for ENNA, ENNA1, ENNB, ENNB1 and BEA.

Hence, an optimal correction for matrix effects and losses during sample preparation was obtained, which was confirmed during method validation.

### Extraction of the Mycotoxins in a Solvent

The next step in the method according to the invention is to extract the mycotoxins contained in the DBS, in a manner as pure and complete as technically feasible. Differently phrased, in this step of the method according to the invention, the aim is to isolate the mycotoxins in the DBS from as much of the other blood constituents as possible. To this end, the method comprises placing the DBS in a test tube, e.g. made of glass, and adding a suitable extraction solvent.

According to a preferred mode of operation, as extraction solvent, use can be made of a mixture of water/acetone/acetonitrile, preferably in the following respective volumes: 30/35/35 v/v/v.

The amount of solvent to be used can vary widely, but preferably an amount ranging from 0.1 up to 10 ml, preferably from 0.5 up to 5 ml, more preferably around 1 ml can be used.

The sample comprising the DBS and the extraction solvent is subjected to an ultrasonic treatment, preferably during a period of time ranging from 15 to 45 minutes, preferable around 30 minutes.

Upon completion of the ultrasonic bath step, the solvent is poured over in another test tube; the remaining saver card paper can be removed.

### Drying and Reconstitution of the Solvent

The aim of the present step is to prepare a sample for injection into the liquid chromatography (LC) apparatus. This implies that the DBS sample should be further treated in view of the detectability as well in qualitative as in quantitative manner of the mycotoxins in the sample in the LC-MS/MS or LC-HRMS.

The latter can be realized on the one hand by an appropriate selection of the reconstitution solvent, and on the other hand by determining the appropriate amount of such reconstitution solvent to be used.

In this stage of the method of the present invention, the extraction solvent, containing the mycotoxins and related metabolites to be detected, is dried, e.g. under a gentle nitrogen stream at a suitable temperature, e.g. between 20 and 60° C., preferably between 30 and 50° C., more preferably at around 40° C.+/−5° C.

Upon drying, the dried material is reconstituted in a solvent, suitable for injection into the LC apparatus.

The inventors have found that to that end, according to the method of the invention, a solvent consisting of water/methanol/formic acid preferably can be used. Preferably the dried material is reconstituted in an appropriate amount of such solvent, being 60 μal (this amount corresponding to the same amount of the initial DBS, drop of blood). By selecting the same amount of blood as of the original DBS, the concentration of mycotoxins in the sample to be analyzed is the same as in the original blood drop.

### Experimental Support:

The following experiments, performed by the inventors, illustrates the above. Three different reconstitution solvents (n=3 per condition) were evaluated: water/ACN/acetic acid (AA) (95/5/0.1, v/v/v); water/methanol/formic acid (60/40/0.1, v/v/v) and water/methanol (15/85, v/v). The first two mixtures have previously been applied for the detection of multiple mycotoxins or OTA alone in human DBS. The latter has been used by co-inventors Lauwers et al. in an LC-MS/MS method for multi-mycotoxin determination in pig and chicken plasma. The best results were obtained using methanol instead of acetonitrile in the reconstitution solvents (The FIGURE). Next, calibration curves were made with the two methanol containing reconstitution solvents and the lowest concentration achieved for each component as well as the peak shape were compared. The mixture of water/methanol/formic acid (60/40/0.1, v/v/v) showed the best results for both parameters and was therefore chosen as the preferred solvent. The same findings were also observed for DBS obtained from broiler chickens.

The FIGURE shows the evaluation of the analyte peak areas after LC-MS/MS analysis of DBS (60 μl), extracts which were re-dissolved in three different reconstitution solvents: water/acetonitrile (ACN)/acetic acid (AA) (95/5/0.1, v/v/v); water/methanol (MeOH)/formic acid (FA) (60/40/0.1, v/v/v) and water/MeOH(15/85, v/v). Individual mycotoxins were spiked in whole blood at a concentration of 10 ng·ml⁻¹. The mean (n=3) LC-MS/MS peak areas+standard deviation (SD) are shown in graph.

The following mycotoxins were the subject of the experiments:

Deoxynivalenol (DON), de-epoxy-deoxynivalenol (DOM1), 3/15-acetyl deoxynivalenol (3/15ADON), aflatoxin B1 (AFB1), aflatoxin M1 (AFM1), enniatin A (ENNA), enniatin A1 (ENNA1), enniatin B (ENNB), enniatin B1 (ENNB1), beauvericin (BEA), fumonisin B1 (FB1), fumonisin B2 (FB2), ochratoxin A (OTA), zearalenone (ZEN), α-zearalenol (AZEL), β-zearalenol (BZEL), α-zearalanol (AZAL), β-zearalanol (BZAL), zearalanone (ZAN), tenuazonic acid (TEA), alternariol (AOH), alternariol mono-methyl-ether (AME), T2 toxin (T2).

### LC (Liquid Chromatography)

The next step of the method of the present invention is the qualitative and quantitative determination of the various mycotoxins and related phase I and II metabolites comprised in the blood sample.

This can be accomplished by subjecting the dried and reconstituted DBS sample to a separation by liquid chromatography. As set forth supra, such apparatus comprises a column comprising a stationary phase and a mobile (liquid) phase. In the column of such apparatus the mycotoxins and related metabolites are separated on the basis of their different affinity to on the one hand the stationary (or fixed) phase of the column, and on the other hand the mobile or liquid phase of such column. Given such differences in affinity, the residence time and hence the transfer time of the various mycotoxins and related metabolites through the column of the apparatus will be different, in term resulting in an appropriate separation of the individual mycotoxins.

A column suitable for use in the method of the present invention comprises HSS (High Strength Silica) Technology particles available from Waters® and its Benelux dealer, LabMakelaar Benelux BV Knibbelweg 18 C, Zevenhuizen, Nederland, e.g. the 1.8 μm High Strength Silica particle.

According to a preferred embodiment of the present invention, the composition of the liquid phase passing through the column of the liquid chromatography apparatus, is varied over time, according to the time of injection of the liquid phase into the column. In a preferred mode of operation, four different modes can be used, two positive and two negative.

At the start of the operation, the mobile phase comprises predominantly water (e.g. up to 95%) and a small proportion of acetonitrile (e.g. up to 5%), and as time goes by, the composition of the liquid phase is changed, whereby the amount of water gradually decreases (e.g. to approximately 5%) and the amount of acetonitrile gradually increases (e.g. up to approximately 95%). This is called the gradient.

As a result of the above, the various individual mycotoxins and related metabolites will exit the liquid chromatography column one after the other; at such stage, these compounds are suitable for being detected in the subsequent stage of the present invention, by the MS/MS or HRMS apparatus.

### Experimental Support:

Four different reversed phase columns (Hypersil Gold 50 mm×2.1 mm, dp: 1.9, Thermo Scientific, Breda, The Netherlands; Zorbax Eclipse C18 50 mm×2.1 mm, dp: 1.8, Agilent, Sint-Katelijne-Waver, Belgium; Acquity BEH-C18 50 mm×2.1 mm, dp: 1.7, Waters, Milford, Mass., USA; and Acquity HSS-T3 100 mm×2.1 mm, dp: 1.8, Waters, Milford, Mass., USA) were tested to achieve chromatographic separation of the selected mycotoxins. The best separation of all components was obtained on the HSS-T3 column. The abbreviation HSS stands for High Strength Silica, the term T3 stands for a trifunctional C₁₈ alkyl chain.

The latter column is available as Acquity UPLC HSS T3 column from Waters Corporation, 34 Maple Street, Milford, USA.

For further details regarding the chromatography apparatus suitable for use in the method of the present invention, reference is made to Document (1) as identified supra, paragraph 4.5, page 23 of 30, the content whereof is incorporated herein by reference.

### Detection of Mycotoxins by MS/MS and HRMS

For the detection of the mycotoxins separated by the LC apparatus, two steps are performed.

The first step is using the LC-MS/MS method and apparatus. When this technique is used, the detection is performed on the basic (‘precursor’) molecule in the first MS stage, as well as on some fragments of such molecule in the second MS stage.

In the LC-HRMS method, the fragments are not the subject of the analysis, only the precursor molecule is the subject of the analysis. In this application the molecular mass of the subject molecule to be detected, can be determined with utmost precision, so enabling an accurate detection of the molecule itself.

For further details regarding the MS/MS apparatus suitable for use in the method of the present invention, reference is made to Document (1) as identified supra, paragraph 4.6.1, page 23 of 30, the content whereof is incorporated herein by reference.

For further details regarding the HRMS apparatus suitable for use in the method of the present invention, reference is made to Document (1) as identified supra, paragraph 4.6.2, page 24 of 30, the content whereof is incorporated herein by reference.

### Method Validation:

Upon completion of the development of the method according to the present invention, the inventors have undertaken the task of validating the method according to the present invention with respect to the DBS from pigs and broiler chickens.

For the purpose of validation, calibration curves have been set up, on the one hand with respect to the DBS from pigs, on the other hand with respect to the DBS from broiler chickens, in both cases for each of the 24 mycotoxins and their phase I metabolites as set forth above.

To that end, tests have been run on three different days as well within a day, and the mean values have been calculated based on these results.

The results of these tests are described hereinafter.

Reference is hereby made to Document (2) as identified supra, paragraph 2.2, page 5 of 22, the content whereof is incorporated herein by reference.

The linearity expressed as a correlation coefficient (r) and the goodness-of-fit (g) is shown in Table 1 for pig DBSs and Table 2 for chicken DBSs and is the mean±standard deviation of three curves across three different days of analysis. The r ranged between 0.991 and 0.999 for pigs and between 0.993 and 0.998 for broiler chickens. The g-value varied from 6% to 17% in pig and 8% to 19% in broiler chicken DBSs. Most of the calibration curves matched a linear model with a 1/x weighing factor, except for the ENNs and BEA which are best described by a quadratic model with a 1/x weighing factor. For most mycotoxins, the linearity ranged between 0.5 and 200 ng·ml⁻¹. However, the limit of quantification (LOQ) and thus the lowest concentration in the calibration curve was set at 1 ng·ml⁻¹ for ZEN, AZAL, BZEL, ZAN, DOM1 and AME in pigs and for ZEN, AZAL, BZAL, ZAN, AOH, T2, ENNA and FB1 in broiler chickens. In broiler chickens, AZEL had an LOQ of 4 ng·ml⁻¹ and FB2 2 ng·ml⁻¹ and in pigs AOH had an LOQ of 10 ng·ml⁻¹. The results for the LOQ and corresponding LOD values are found in Table 1 and Table 3, respectively.

The within-day and between-day precision and accuracy at a concentration of 10 and 100 ng·ml⁻¹ met the requirements for all mycotoxins for both species. The results of the within-day and between-day precision and accuracy experiments can be found in Table 3 and Table 4, respectively.

The results for signal suppression or enhancement (SSE) after the extraction of DBSs from broiler chickens and pigs showed acceptable results (range 60-112%) for most components. Moreover, in pig DBSs, good extraction recovery rates were also observed. For some components, SSE was more pronounced and extraction recovery was rather low (<60%). However, for all mycotoxins, an adequate internal standard (IS) and matrix-matched calibration curves were used, resulting in validation results for accuracy and precision matching the acceptance criteria. The results for matrix effects and extraction recovery are shown in the Tables set forth is said publication.

For the sake of completeness, we reproduce hereinafter the table regarding the test results for pig whole blood:

TABLE 1
Validation results for linearity shown as the mean ± standard
deviation of three curves across three different days of
analysis (linear range, correlation coefficient (r) and
goodness-of-fit coefficient (g)), limit of quantification
(LOQ) and limit of detection (LOD) of 23 my
cotoxins in dried blood spots of pig whole blood.

| | Linearity (n = 3 Different | | | |
|---|---|---|---|---|
| | Days) Linear Range | | LOQ | LOD |
| Analyte | (ng · ml−1) | r ± SD | ng · mL⁻¹ | ng · mL⁻¹ |
| ZEN | 1-200 | 0.999 ± | 1.0 | 0.09 |
| | | 0.001 | | |
| AZEL | 0.5-200 | 0.998 ± | 0.5 | 0.20 |
| | | 0.000 | | |
| AZAL | 1-200 | 0.998 ± | 1.0 | 0.38 |
| | | 0.001 | | |
| BZAL | 0.5-200 | 0.997 ± | 0.5 | 0.11 |
| | | 0.001 | | |
| BZEL | 1-200 | 0.994 ± | 1.0 | 0.21 |
| | | 0.004 | | |
| ZAN | 1-200 | 0.996 ± | 1.0 | 0.10 |
| | | 0.004 | | |
| TEA | 0.5-200 | 0.996 ± | 0.5 | 0.04 |
| | | 0.003 | | |
| AOH | 10-200 | 0.994 ± | 1.0 | 0.74 |
| | | 0.005 | | |
| AME | 1-200 | 0.993 ± | 1.0 | 0.01 |
| | | 0.002 | | |
| DON | 0.5-200 | 0.994 ± | 0.5 | 0.11 |
| | | 0.003 | | |
| DOM1 | 1-200 | 0.997 ± | 1.0 | 0.23 |
| | | 0.002 | | |
| 3/15AD | 0.5-200 | 0.994 ± | 0.5 | 0.06 |
| ON | | 0.002 | | |
| T2 | 0.5-200 | 0.996 ± | 0.5 | 0.01 |
| | | 0.004 | | |
| AFB1 | 0.5-200 | 0.994 ± | 0.5 | 0.001 |
| | | 0.002 | | |
| AFM1 | 0.5-200 | 0.991 ± | 0.5 | 0.01 |
| | | 0.002 | | |
| OTA | 0.5-200 | 0.995 ± | 0.5 | 0.01 |
| | | 0.003 | | |
| ENN A1 | 0.5-200 | 0.997 ± | 0.5 | 0.05 |
| | | 0.002 | | |
| ENNA | 0.5-100 | 0.998 ± | 0.5 | 0.01 |
| | | 0.001 | | |
| ENNB | 0.5-100 | 0.999 ± | 0.5 | 0.001 |
| | | 0.000 | | |
| ENNB1 | 0.5-100 | 0.998 ± | 0.5 | 0.001 |
| | | 0.001 | | |
| BEA | 0.5-200 | 0.997 ± | 0.5 | 0.02 |
| | | 0.003 | | |
| FB2 | 0.5-200 | 0.996 ± | 0.5 | 0.35 |
| | | 0.002 | | |
| FB1 | 1-200 | 0.994 ± | 0.5 | 0.23 |
| | | 0.004 | | |

For the sake of completeness, we reproduce hereinafter the table regarding the test results for broiler chicken whole blood:

TABLE 2
Validation results for linearity shown as the mean ±
standard deviation of three
curves across three different days of analysis
(linear range, correlation
coefficient (r) and
goodness-of-fit coefficient (g)), limit of quantification
(LOQ) and limit of detection (LOD) of
23 mycotoxins in dried blood spots of
broiler chicken whole blood.

| | Linearity (n = 3 Different Days) | | | | |
|---|---|---|---|---|---|
| | Linear | | | | |
| | Range | | | LOQ | LOD |
| Ana- | (ng · | | | ng · | ng · |
| lyte | ml-1) | r ± SD | g ± SD | mL⁻¹ | mL⁻¹ |
| ZEN | 1-200 | 0.997 ± 0.001 | 17 ± 3 | 1.0 | 0.12 |
| AZEL | 4-200 | 0.997 ± 0.002 | 8 ± 3 | 4.0 | 1.10 |
| AZAL | 1-200 | 0.997 ± 0.002 | 16 ± 6 | 1.0 | 0.15 |
| BZAL | 1-200 | 0.996 ± 0.003 | 18 ± 2 | 1.0 | 0.27 |
| BZEL | 0.5-200 | 0.996 ± 0.003 | 16 ± 2 | 0.5 | 0.1 |
| ZAN | 1-200 | 0.997 ± 0.001 | 13 ± 5 | 1.0 | 0.21 |
| TEA | 0.5-200 | 0.998 ± 0.001 | 12 ± 6 | 0.5 | 0.001 |
| AOH | 1-200 | 0.996 ± 0.002 | 17 ± 3 | 1.0 | 0.02 |
| AME | 0.5-200 | 0.997 ± 0.001 | 17 ± 4 | 0.5 | .001 |
| DON | 0.5-200 | 0.997 ± 0.000 | 15 ± 1 | 0.5 | 0.18 |
| DOM1 | 0.5-200 | 0.998 ± 0.001 | 17 ± 1 | 0.5 | 0.16 |
| 3/15 | 0.5-200 | 0.995 ± 0.003 | 16 ± 4 | 0.5 | 0.09 |
| ADON | | | | | |
| T2 | 1-200 | 0.998 ± 0.000 | 13 ± 5 | 1.0 | 0.03 |
| AFB1 | 0.5-200 | 0.997 ± 0.001 | 15 ± 4 | 0.5 | 0.01 |
| AFM1 | 0.5-200 | 0.997 ± 0.001 | 15 ± 2 | 0.5 | 0.01 |
| OTA | 0.5-200 | 0.998 ± 0.001 | 19 ± 1 | 0.5 | 0.05 |
| ENN | 0.5-200 | 0.993 ± 0.002 | 14 ± 5 | 0.5 | 0.04 |
| A1 | | | | | |
| ENNA | 1-200 | 0.995 ± 0.003 | 16 ± 2 | 1.0 | 0.11 |
| ENNB | 0.5-200 | 0.994 ± 0.001 | 12 ± 1 | 0.5 | 0.07 |
| ENNB1 | 0.5-200 | 0.998 ± 0.002 | 12 ± 3 | 0.5 | 0.001 |
| BEA | 0.5-200 | 0.996 ± 0.000 | 15 ± 4 | 0.5 | 0.07 |
| FB2 | 1-200 | 0.997 ± 0.001 | 16 ± 3 | 1.0 | 0.96 |
| FB1 | 2-200 | 0.995 ± 0.001 | 15 ± 3 | 2.0 | 1.87 |

The advantages of the method according to the present invention:

As compared to monitoring the animal feed, the present invention offers the following advantages:
1) Biomonitoring measures the exposure at an individual level while feed analysis shows a general risk. Exposure at an individual level takes variation in food consumption and ADME parameters into account and integrates contamination from all different sources leading to a more accurate estimation and possible use in toxicokinetic studies. (ADME stands for Absorption, distribution, metabolism, excretion, all of these toxicokinetic parameters describing the effect of the animal body on the toxins concerned,)
2) Although feed is an accessible matrix, it is difficult to sample correctly due to the presence of hotspots. Urine, feces and in particular plasma/blood in contrast are easy to sample correctly and also allow sequential sampling.
3) Measuring complete exposure: modified mycotoxins can convert back to their original forms during digestion and so contribute to the overall effect of the toxins. Biomonitoring includes these effects and gives thus a very accurate estimation of the risk.

### Biomonitoring Analysis as a New Tool to Evaluate Mycotoxin Detoxifiers:

A common way to counter the harmful effect of mycotoxins comprises adding mycotoxins detoxifiers to the animal feed. An example of such detoxifier is the product marketed by Innovad N.V., Cogels-Osylei 33, 2600 Antwerp, Belgium, under the brand name Escent S®. A detoxifier product is a product that diminishes the effects associated with toxins.

A possible way to prove the efficacy of these products is doing in vivo absorption tests. In these trials the concentration of mycotoxins in animal matrices is detected and the difference in concentration with and without detoxifier is determined. This difference is a measure for the efficacy of the product. This type of test gives accurate results and should in the future replace in vitro tests or in vivo test based on non-specific parameters such as growth performance and feed conversion.

1. Method for the detection of one or more mycotoxin(s), one or more of their phase I and/or one or more of their phase II metabolites in broiler chickens or pigs, the method comprising:
collecting the blood of broiler chickens or pigs as a dried blood sample;
preparing the dried blood sample for analysis;
analyzing the prepared dried blood sample by liquid chromatography-tandem mass spectrometry and high-resolution mass spectrometry.

2. Method according to claim 1 for the detection of:
one or more mycotoxins selected from the following list of 17 parent mycotoxins:
Deoxynivalenol,
3/15-acetyldeoxynivalenol,
T2-toxin,
HT-2 toxin,
Aflatoxin B1,
Ochratoxin A,
Enniatin A1,
Enniatin A,
Enniatin B,
Enniatin B1,
Beauvericin,
Fumonisin B1,
Fumonisin B2,
Tenuazonic acid,
Alternariol,
Alternariol methyl ether,
Zearalenone,
one or more phase I metabolites selected from the following list of 7 phase I metabolites:
De-epoxy-deoxynivalenol,
Aflatoxin M1,
α-Zearalenol,
α-Zearalanol,
β-Zearalanol,
β-Zearalenol,
Zearalanone,
one or more phase II metabolites selected from the following list of 5 phase II metabolites:
D eoxynivalenol-glucuronide,
D eoxynivalenol-sulphate,
Zearalenone-glucuronide,
α-zearalenol-glucuronide,
β-zearalenol-glucuronide.

3. Method according to claim 2, whereby the mycotoxin(s) and the phase I metabolites are detected by liquid chromatography-tandem mass spectrometry and the phase II metabolites are detected by high resolution mass spectrometry.

4. Method according to claim 1, whereby collecting the dried blood sample comprises collecting a drop of blood on a filter paper, followed by drying at room temperature.

5. Method according to claim 4, whereby collecting the dried blood sample comprises isolating the dried blood sample from the filter paper by punching out a paper disk out of the filter paper, preferably round and about 8 mm in diameter, using a biopsy punch.

6. Method according to claim 5, comprising, prior to analyzing, extracting the mycotoxins and the phase I and II metabolites from the dried blood sample in an extraction solvent and subjecting the extraction solvent and the dried blood sample to an ultrasonic bath treatment.

7. Method according to claim 6, whereby the extraction solvent comprises a water/acetonitrile/acetone mixture.

8. Method according to claim 6, comprising drying the extraction solvent and reconstituting the dried mass in a reconstitution solvent.

9. Method according to claim 8, whereby the reconstituting solvent comprises a water/methanol/formic acid mixture.

10. Method according to claim 1, comprising extracting the collected dried blood sample and mycotoxins, phase I and phase II metabolites in an extraction solvent, drying and reconstituting the extracted dried blood sample in a reconstitution solvent, and analyzing the reconstituted dried blood sample by liquid chromatography-tandem mass spectrometry and liquid chromatography-high resolution mass spectrometry.

11. Method according to claim 10, comprising analyzing the reconstituted dried blood sample in a two subsequent steps:
on the one hand, analyzing by liquid chromatography-tandem mass spectrometry, the mass spectrometer apparatus operating in negative electro-spray ionization mode, and using as mobile phase, a mixture of acetic acid, water and acetonitrile, the proportion of acetonitrile gradually increasing during the liquid chromatography process, one or more of the following mycotoxins: zearalenone, zearalanone, α-zearalenol, α-zearalanol, β-zearalanol, β-zearalenol, tenuazonic acid, alternariol, alternariol methyl ether;
on the other hand, analyzing by liquid chromatography tandem mass spectrometry, the mass spectrometer apparatus operating in positive electro-spray ionization mode, and using as mobile phase in the liquid chromatography column of the liquid chromatography tandem mass spectrometry method, a mixture of ammonium formate, formic acid, water and methanol, the proportion of methanol gradually increasing during the liquid chromatography process, one or more of the following mycotoxins:
deoxynivalenol (DON), de-epoxy-deoxynivalenol (DOM1), 3/15-acetyldeoxynivalenol (3/15ADON), T2-toxin (T2), HT-2 toxin (HT2), aflatoxin B1 (AFB1), aflatoxin M1 (AFM1), ochratoxin A (OTA), enniatin A1 (ENNA1), enniatin A (ENNA), enniatin B (ENNB), enniatin B1(ENNB1), beauvericin (BEA), Fuminisin B1, Fumonisin B2.

12. Method according to claim 1, further comprising detecting the mycotoxin(s) and the phase I metabolites of such mycotoxins by subjecting the collected blood sample to liquid chromatography-tandem mass spectrometry, followed by detecting the phase II metabolites of the mycotoxins to liquid-chromatography-high resolution mass spectrometry.

13. Method according to claim 1, further comprising spiking the collected dried blood sample with one or more internal standards.

14. Method according to claim 12, comprising spiking the collected dried blood sample with one or more internal standards selected from the following list:
¹³C₁₅-deoxynivalenol,
¹³C₁₇-aflatoxin B1,
¹³C₂₀-ochratoxin A,
¹³C₂₄-T2-toxin,
¹³C₃₄-fumonisin B1,
¹⁵N₃-enniatin B,
¹³C₆¹⁵N-tenuazonic acid,
¹³C₁₈-zearalenone.

15. Use of the method of claim 1 for the assessment of the exposure of pigs or broiler chickens to feed contaminated with mycotoxins.

16. Use of the method of claim 1 for assessing the impact of the addition of mycotoxin detoxifying agents to animal feed.
